(19) United States
(12) Patent Application Publication
(10) Pub. No.: US 2003/0024533 A1
(43) Pub. Date: Feb. 6, 2003

(54) Multi-port mask

(76) Inventor: Joseph A. Sniadach, Baltimore, MD (US)

Correspondence Address: Arstrong Westerman & Hattori LLP, 502 Washington Avenue, Suite 220, Townson, MD, 21204, US

(21) Appl. No.: 09/920,695

(22) Filed: Aug. 2, 2001

(51) Int. Cl. A62B 1/00; A62B 18/02; A62B 18/08; A61M 16/00

(52) U.S. Cl. 128/205.25; 128/206.21; 128/206.28; 128/206.29; 128/205.13; 128/202.28

(57) ABSTRACT

A mask with multi-ports is set forth allowing many functions to take place while the mask is affixed to the patient's face covering both the nose and mouth. For example, the mask allows for patient ventilation while at the same time there is provided a port for instrument access and a port for fluid suction. The multi-port mask may have attached thereto a reservoir bag.

### FIELD OF THE INVENTION

The herein disclosed invention finds applicability in the medical field and particularly in applications wherein oxygen is needed to reach the lungs. In particular, the field of the invention is medical masks employed to deliver oxygen or some other type of gas.

### BACKGROUND OF THE INVENTION

There is currently a very serious need in the medical profession for a multi-use oxygenating mask which would allow the physician to perform diagnostic and therapeutic procedures safely by maintaining adequate oxygenation and ventilation concomitantly. The oxygenating mask of this invention would perform these functions based on a new design. This oxygenating mask would have components of an inflatable base to improve facial seal and thus oxygenation. The mask would also have two exhalation ports with one-way valves which allow adequate ventilation, and features a port with a penetrated cover through which an instrument can be passed and still allow for oxygen retention. The mask has an oxygen port for direct flow of wall or cylinder oxygen and also maintains a covered port which can be utilized to assist ventilation should over-sedation respiratory depression occur. Additional features include a slide-port to allow for suctioning of blood or secretions while oxygenation is maintained and the medical procedure is uninterrupted. Finally, a port with an oxygen reservoir bag would also be available to access should oxygenation requirements dramatically increase.

Currently there is no mask available that offers all of these features. The herein disclosed mask brings together what the inventor, who is an expert in airway management, deems to be essential features needed to allow the practitioner to maintain adequate patient oxygenation and ventilation and at the same time perform auxiliary medical procedures which would include but not be limited to transesophageal echocardiogram, bronchoscopy or esophagogastroduodenoscopy. Without such oxygenation and ventilation, hypoxia, acidosis and electrolyte imbalance would ensue, leading to respiratory failure and arrest, cardiac arrest, heart damage, or brain injury. The inventor having participated in therapeutic/diagnostic procedures requiring monitored sedation, has noticed many very significant health hazards from required-medications, resulting in over-sedation and also resulting in respiratory and circulatory failure which require emergency airway intervention. The purpose of the mask of this invention is to avoid many of those problems encountered by those skilled in the art.

Current oxygen sources and techniques of administration include:

1. Nasal cannula (nasal prongs)—allowing for easy access to the mouth but is considered a low flow system with valuable oxygen lost to the atmosphere. High oxygen levels, especially when needed cannot be achieved.

2. Non-rebreather face mask—allows for high flow, high concentration oxygen but this is again problematic when O₂ escapes into the atmosphere when lifting the mask in order to gain access into the mouth.

3. Standard issue anesthesia mask—allows for high flow, high concentration of oxygen, but has no other ports to allow for access of instruments, while maintaining oxygen concentration. The mask must be used with an anesthesia machine/circuit that allows for CO₂ elimination since only one port exists for inhalation/exhalation.

4. Patil-Syracuse® mask—This mask is a modified standard issue anesthesia mask adding with an additional port added to allow for fiberoptic guided tracheal intubation while administering sedative medications. Again, similar to the standard issue mask, only one other port is present in which an anesthesia circuit is connected performing both oxygenation and ventilation (or exhalation).

Prior Art Patent

Hudson in U.S. Pat. No. 2,859,748 teaches a breathing mask with exhalation openings and an opening to receive an oxygen tube. The herein disclosed invention is distinct from that of Hudson in that the Hudson mask fits only over the nose, while the herein disclosed mask fits over both the nose and mouth. Further, Hudson makes no provision for an instrument access port, nor a port for fluid suction.

Advantages of the Multi-Port Oxygenating Mask When Compared with Current Devices:

1. Nasal cannula—The oxygenating mask produces much higher oxygen levels as required during “sedative procedures” in order to prevent heart or brain injury.

2. Non-rebreather face mask—This oxygenating mask produces higher oxygen levels since the non-rebreather for the mask acts like a nasal cannula when lifted to gain access into the mouth.

3. Standard issue anesthesia mask—The oxygenating mask emboding both oxygen ports and exhalation ports can act independently of an anesthesia machine and circuit. In addition, the mask of this invention can be used in a physician's office setting by allowing access into the mouth, as well as removal of blood or secretions during procedures which are lacking in the standard issue anesthesia mask.

4. Patil-Syracus® mask—This mask is similar to the standard anesthesia mask and requires an anesthesia circuit for both inhalation and exhalation since independent ports embodied in the new oxygenating mask of this invention are lacking. These independent ports, along with a back-up port available for resuscitation and the slide opening for blood or secretion removal, without moving the mask, adds novel features lacking in the Patil-Syracuse® mask. Therefore, the design of the Patil-Syracuse® mask does not allow itself to be used in an “office-based” setting by non-anesthesiologist. Furthermore, cost-prohibitive factors must be examined as the PatilSyracuse® mask is expensive, whereas the new oxygenating mask of this invention would be inexpensive and disposable. Cost effectiveness is a significant consideration.

Current Health Threat from Office Sedation Necessitating Usage of the Inventive Muli-Port Oxygenating Mask

Many locations exist in practically every town in America where a single person performs the operation procedure and also administers the sedation and oxygen; i.e., surgery in the operating room without an anesthetist. The surgeon is obviously preoccupied with the procedure and attempting to sedate will leave very little attention focused on the patient's ventilation.

Further, most of these practitioners are not overly experienced with airway management.

The difference between sedation and deep sedation (general anesthesia) can be as little as 1 cc in the syringe, with resultant respiratory and cardiac failure.

Who Would benefit from Using the Multi-Port Oxygenating Mask

1. Cardiologist performing transesophageal echocardiography

2. Pulmonologist performing bronchoscopy

3. Gastroenterologist performing esophagogastroduodenoscopy

4. Head and neck surgeons performing office based oral procedures

5. All dental procedures using the multi-port oxygenating mask which achieves improved oxygenation yet full access to the mouth.

Oxygenating and Dental Oxygenating Mask

These multi-port masks currently do not exist and would offer many advantages relative to the inadequate masks currently available. For example, these multi-port masks allow for high flow, high concentration oxygen required during sedation where respiratory reflexes become obtunded. They allow for critical oxygenation as well as carbon dioxide elimination (even more crucial in patients with underlying heart and lung disease) while surgical procedures are performed. Although these masks can be used the single practitioner (one physician/one dentist), it would also allow a second person the means by which to assist ventilation in a facilitated manner. Attention is also brought to the fact that in keeping with the recommendations set by the American Society of Anesthesiologists, it is required that blood oxygen saturation be constantly measured by pulse oximetry.

### SUMMARY OF THE INVENTION

In its broadest aspect, the invention envisions a multi-port mask having therein multiple ports comprising a medical facial mask covering both the nose and mouth and having therein an oxygen port, an exhalation port, an instrument access port and a suction port. As an alternative embodiment, the mask may also have a reservoir bag attached thereto, and further caps are provided for the ports for use when the ports are not in use. As a special feature, the ports may be supplied with a sliding closure, and more specifically, the suction port is supplied with a sliding closure. An alternative, mask may have an instrument access port, an oxygen port, a universal port for ambu resuscitation bag, an exhalation port, a suction port and a reservoir bag port.

### BRIEF DESCRIPTION OF THE DRAWINGS

FIG. 1 is a view of prior art oxygenating mask.

FIG. 2 is a view of a prior art mask with a reservoir bag attachment.

FIG. 3 is a top plan view of the multi-port oxygenating mask of this invention.

FIG. 4 is a left side view thereof.

FIG. 5 is a right side view thereof.

FIG. 6 is an oral-end view thereof.

FIG. 7 is a nasal-end view thereof.

FIG. 8 is a bottom plan view thereof.

FIG. 9 is a cross-section thereof taken along line 9-9 of FIG. 3.

FIG. 10 is sectional view of slide closure suction port taken along line 10-10 of FIG. 5.

FIG. 11 is a view depicting the mask as it would be used in practice. Shown in dashed lines is the head of the patient.

FIG. 12 is a view depicting an alternative embodiment of a multi-port mask.

### DESCRIPTION

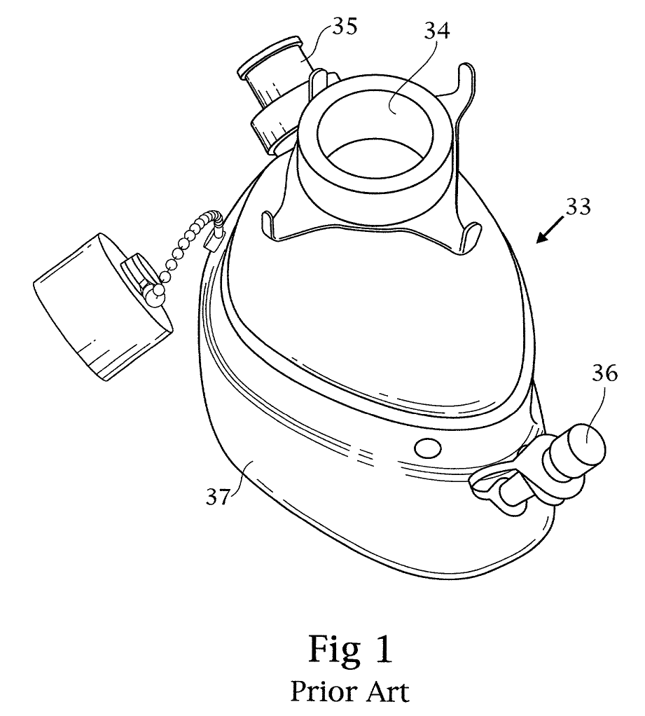

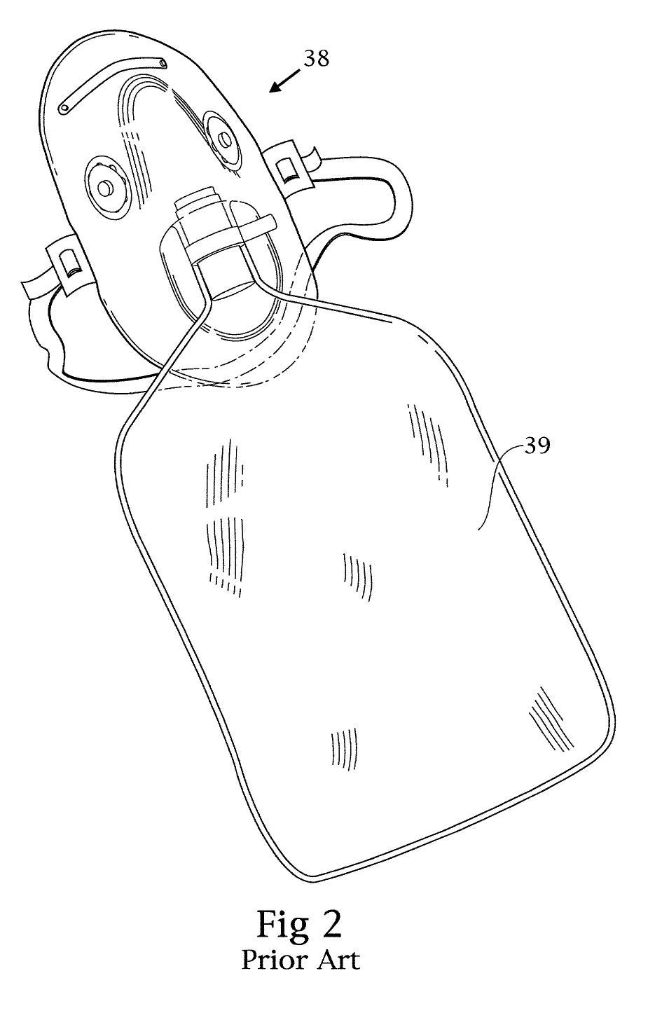

With reference to FIG. 1 a prior art mask, Patil-Syracuse® mask is shown; and with reference to FIG. 2, a reservoir mask is shown. In FIG. 1, the mask 33 has a ventilation tube opening 34, a fiber-optic opening 35 and a fill-valve 36 for the cuff 37. In FIG. 2, the mask 38 with reservoir bag 39 is shown. The masks 33 and 38 are conventional in the art and do not have the requisite number of openings required by the multi-port masks of this invention. The prior art oxygenating masks are deficient in not showing multi-ports as described by this invention for airway access, suction access or instrument access.

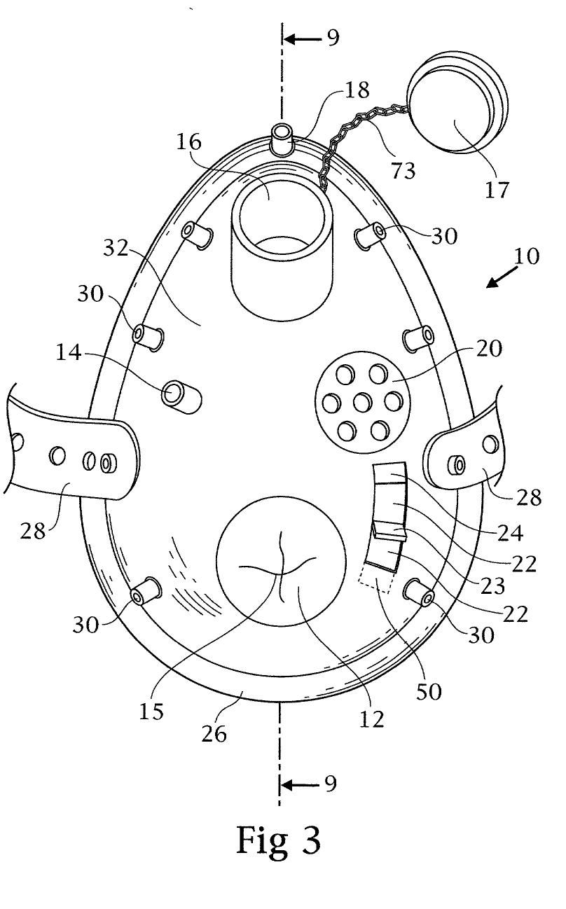

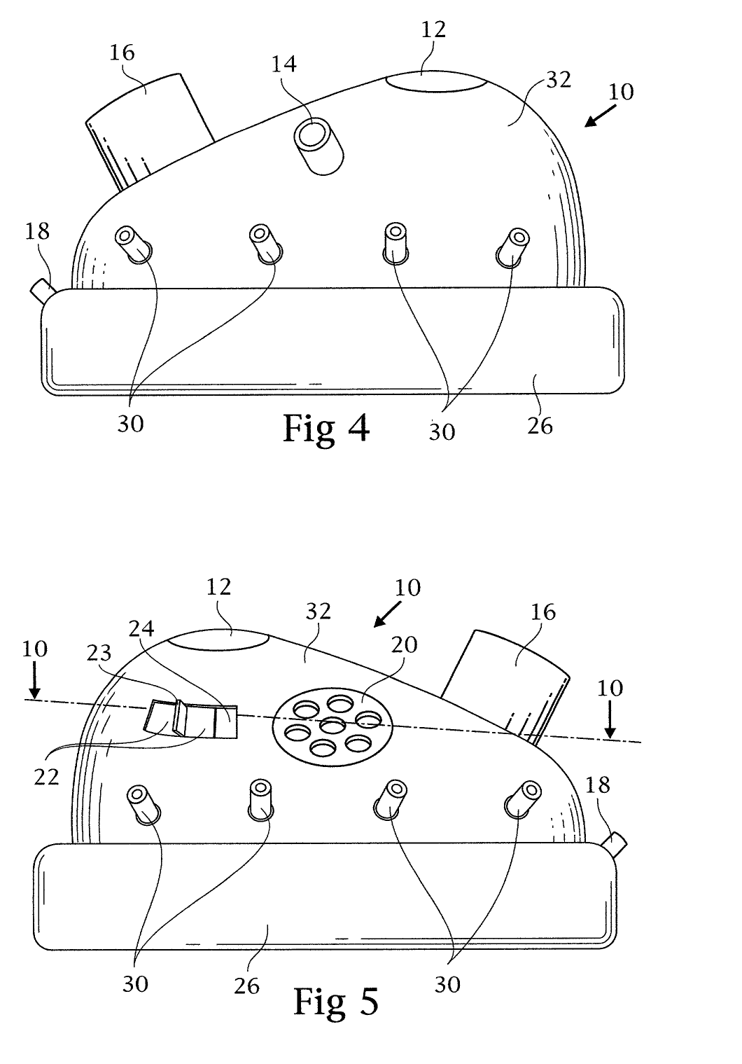

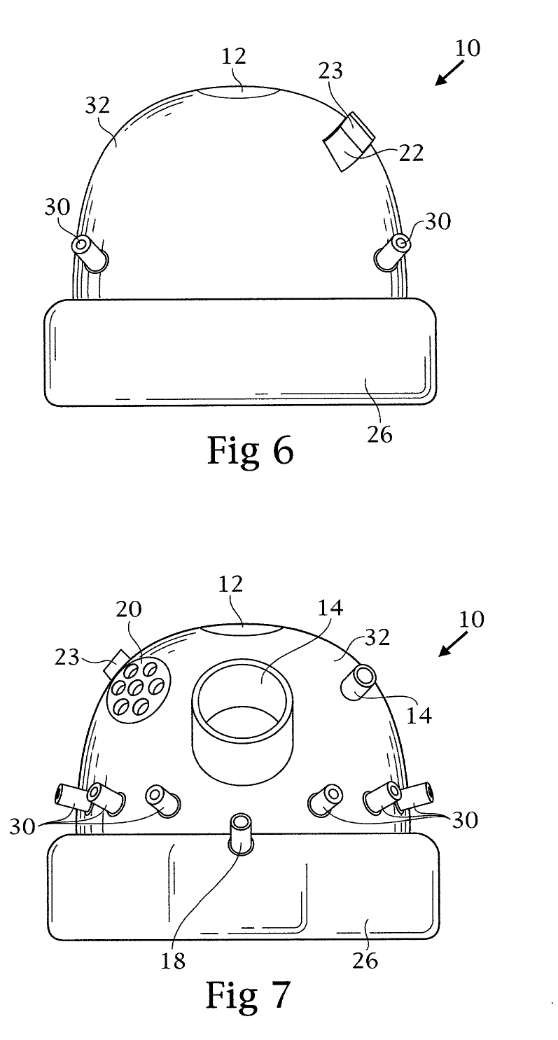

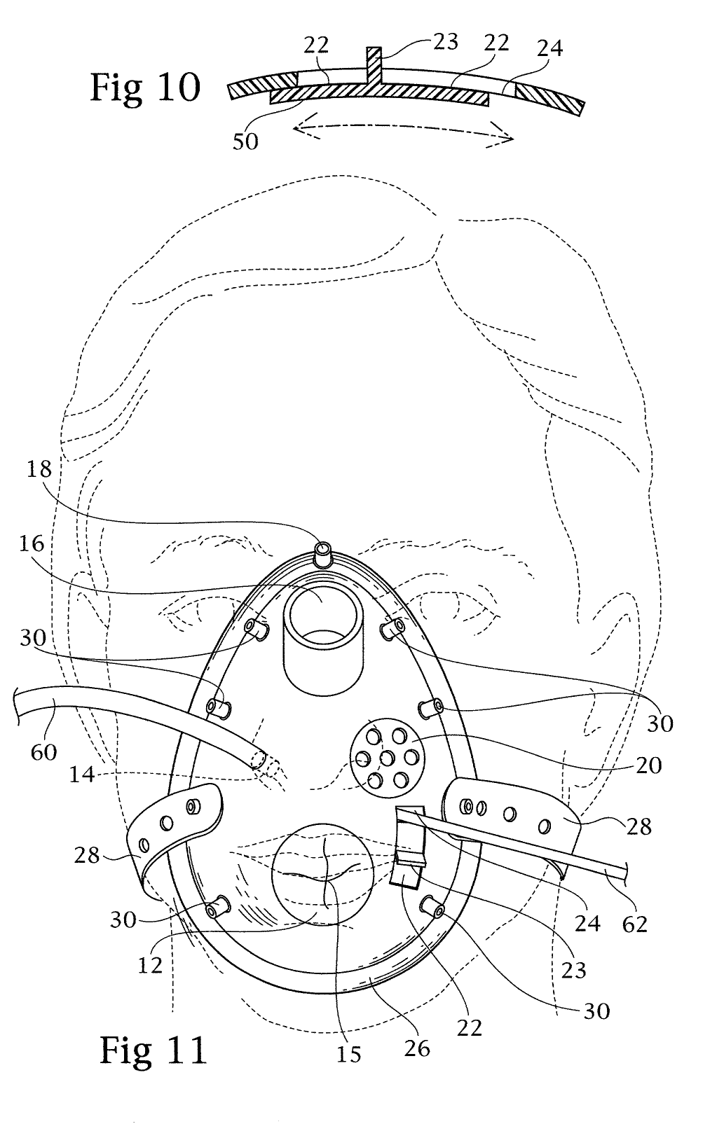

With reference to FIGS. 3 to 11, a multi-port mask 10 has a port for instrument access 12, with a fenestrated opening 15, oxygen port 14, a universal port for ambu resuscitation bag 16, with cap 17, inflation valve 18, an exhalation port 20, suction port opening 24 covered with sliding closure 22 having a tab 23 to facilitate slide, inflatable cuff 26, securing strap 28 best shown in FIGS. 3 and 11, strap studs 30 and clear plastic face 32. Clear plastic is preferred to allow visualization of critical facial areas.

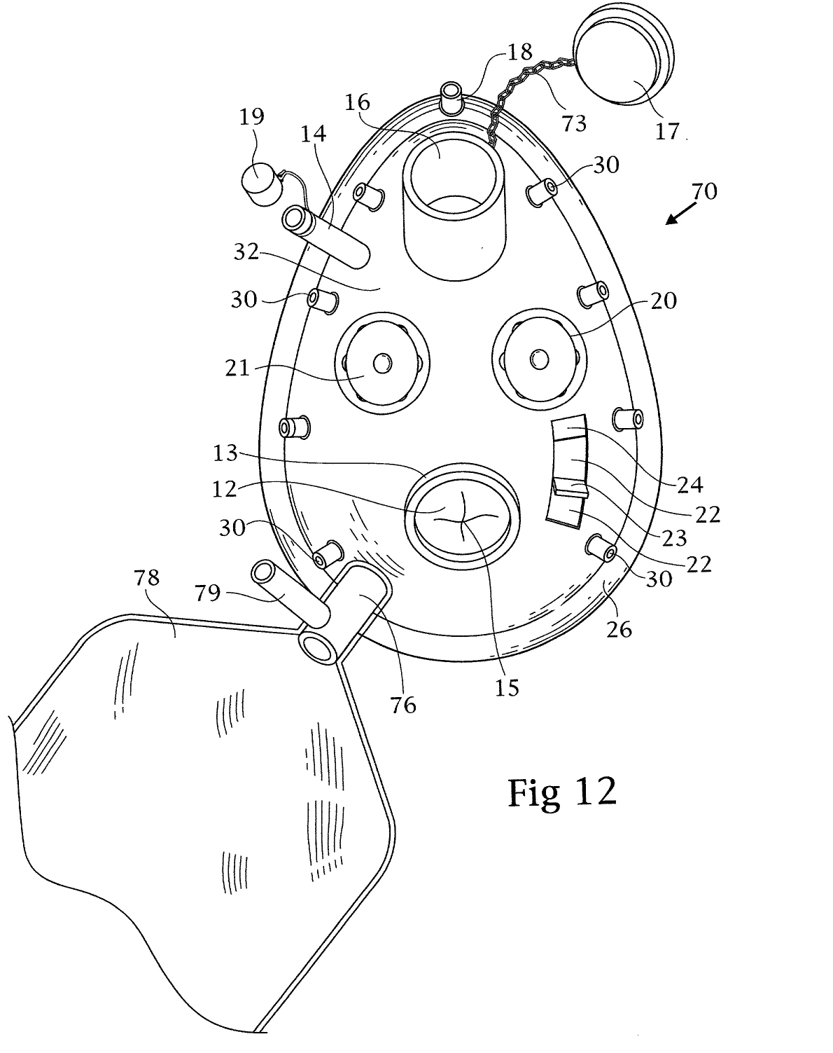

As best shown by FIGS. 3 and 12, various ports can be capped when not needed and oxygen or other gas retained under the mask. Note cap 17 for ambu port 16 and cap 19 for oxygen port nipple 14. Note that the exact position of the oxygen port 14 on the mask is not critical; as note their different positions in FIGS. 3 and 12.

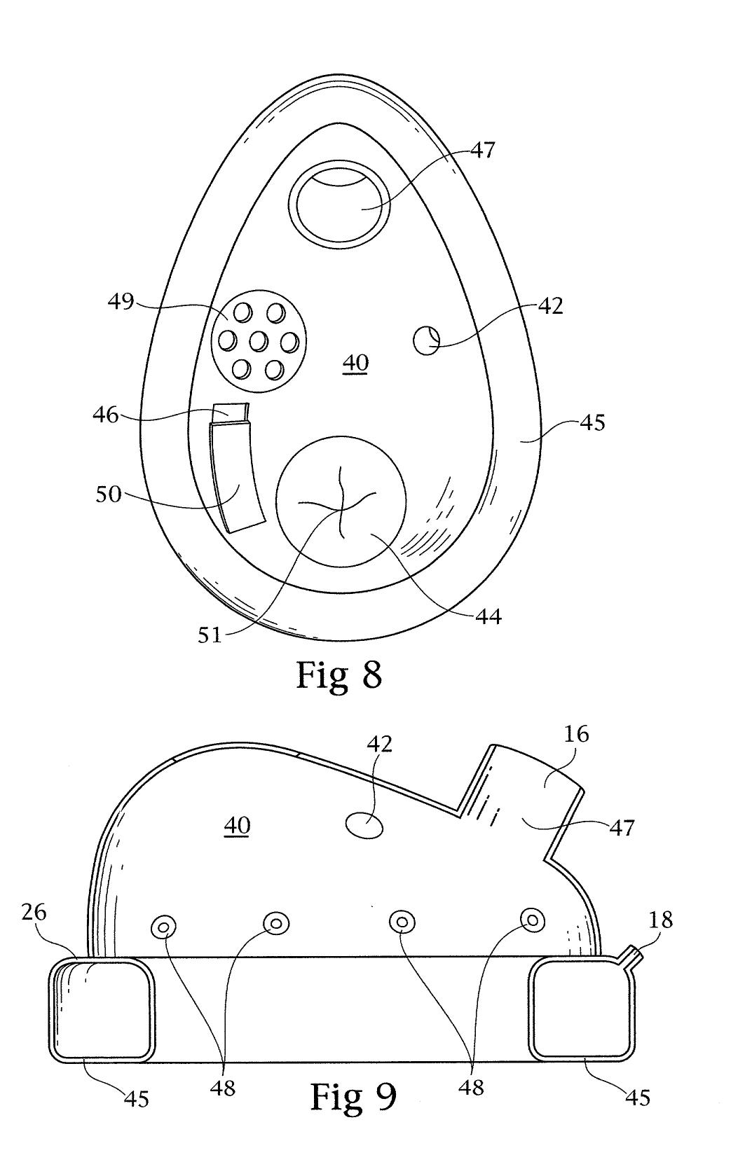

With reference to FIGS. 8 and 9, the underside of the mask 40 shows the underside of the oxygen port 42, the underside of the instrument port 44, the underside of suction port 46, the underside of the ambu port 47, the underside of the strap studs 48, the underside of the inflation cuff 45, the underside of the slide for closure 50, underside of fenestrated opening 15 and the underside of the suction port 46.

In use the mask 10 would be placed over the face of the patient. Straps 28 would be used to snug the mask to the face of the patient; and the cuff 26 would be inflated using inflation valve 18.

With particular reference to FIG. 11, the mask 10 is shown placed over the face of the patient shown in dashed lines. The mask is snugged down over the face of the patient with straps 28 and using inflation valve 18 to inflate cuff 26. Once the mask 10 is in place, the oxygen hose 60, which is attached to an oxygen tank (not shown), can be attached to the oxygen port nipple 14 and oxygen delivery or anesthetic gas delivery started. With proper breathing oxygen, for example, can be delivered by way of tube 60. Exhaled gas is exhausted through exhalation port 20. If tracheal intubation or instrumentation is required, it can be carried out through port 12 and its fenestrated opening 15 for instrument access. In the event fluids have to be suctioned, the procedure can be done through suction port opening 24 using instrument 62.

With reference to FIG. 12, an alternative embodiment of a multi-port mask 70 is described. This mask 70 is shown with a fenestrated opening 15 for easy access of instruments. Cover 17 attached to chain 73 is to be used for ambu port 16. Mask 70, has two exhalation ports 20 and 21. Exhalation ports such as 20 and 21 are known in the art and do not per se form part of the herein disclosed invention. The exhalation ports function by virtue of a rubber flap which rises to exhaust and flaps down to retain air, oxygen or other gas under the mask. FIG. 12 also has a reservoir bag 78 with a portion broken-away for ease of illustration. While reservoir bags per se are known, the reservoir bag in the context of the multi-ports of the inventive mask forms a novel feature of this invention. The reservoir bag 78 can be attached to valve 76 (conventional in the art and schematically shown). Valve 76 using gas entry nipple 79 can be used to fill bag 78 as well as to supply oxygen under the mask 70.

Note that in the alternative embodiment FIG. 12, instrument port 12 and ambu port 16 are provided with collars 13 and 19 for receiving a cap if necessary. The caps are convenient for more efficiently sealing the mask when that port is not in use.

As an example, the over-all dimensions of the multi-port mask are approximately 5½ by 3¾ tapered. It would be obvious to those skilled in the art that the masks of this invention can come in a variety of sizes to accommodate a variety of facial sizes.

There are many advantages attendant to the use of the multi-port mask. For example:

1) Because of the presence of ports for both tank oxygen as well as an ambu bag which can be used by a second person to assist ventilation.

2) The mask has a fenestrated cover through which an instrument can be passed, yet still allow for oxygen retention.

3) The mask has a slide mechanism to suction secretions or blood to improve ventilation while still maintaining oxygenation.

While the ports of the mask have been shown as round or rectangular, the exact configuration is generally not critical. Those skilled in the art could design other configuration without undue experiment. In addition, the alternative placement of ports on the mask could be fashioned by those skilled in the art. For example, the oxygen port could be placed at other convenient locations on the mask.

Obviously, many modifications may be made without departing from the basic spirit of the present invention. Accordingly, it will be appreciated by those skilled in the art that within the scope of the appended claims, the invention may be practiced other than has been specifically described herein.

1. A multi-port mask having therein multiple ports comprising a medical facial mask covering both the nose and mouth and having therein an oxygen port, an exhalation port, an instrument access port and a suction port.

2. The multi-port mask of claim 1 wherein a reservoir bag is attached thereto.

3. The multi-port mask of claim 1 wherein caps are provided for the ports for use when needed.

4. The multi-port mask of claim 1 wherein a port is supplied with a sliding closure.

5. The multi-port mask of claim 1 wherein the suction port is supplied with a sliding closure.

6. The multi-port mask of claim 1 wherein the instrument access port is provided with a fenestrated opening.

7. A multi-port mask having therein various ports comprising a medical facial mask having an instrument access port, an oxygen port, a universal port for ambu resuscitation bag, an exhalation port, and a suction port.

8. A multi-port mask having therein various ports comprising a medical facial mask covering both the nose and mouth and having therein an oxygen port, an exhalation port, an instrument access port, a suction port and a port for a reservoir bag.

9. The multi-port mask of claim 8 wherein there is a reservoir bag attached to a port for receiving the reservoir bag.

10. The multi-port mask of claim 8 being provided with a universal port for ambu resuscitation bag.
